(19) United States
(12) Patent Application Publication
(10) Pub. No.: US 2019/0374659 A1
(43) Pub. Date: Dec. 12, 2019

(54) SYSTEM AND METHOD FOR IMAGING MACROPHAGE ACTIVITY USING DELTA RELAXATION ENHANCED MAGNETIC RESONANCE IMAGING

(71) Applicants: Timothy James Scholl, London (CA); Synaptive Medical Inc., Toronto (CA)

(72) Inventor: Timothy James Scholl, London (CA)

(21) Appl. No.: 16/542,779

(22) Filed: Aug. 16, 2019

Related U.S. Application Data
(63) Continuation-in-part of application No. 15/534,037, filed on Jun. 8, 2017.

(51) Int. Cl. A61K 49/18; A61B 5/055; A61K 49/06; G01R 33/56; G01R 33/48

(52) U.S. Cl. CPC A61K 49/1863; A61B 5/055; G01R 33/4816; A61K 49/1896; G01R 33/5601; A61K 49/06

(57) ABSTRACT

A magnetic resonance imaging (MRI) system is provided for imaging immune response of soft tissue to therapy by, prior to therapy, administering a contrast agent to the soft tissue; imaging a region of interest using delta relaxation enhanced magnetic resonance (DREMR) to define a functional section; selectively sampling local cells in the functional section; conducting immuno-assay analysis on the sampled local cells; and following therapy, further imaging said region of interest using DREMR to assess immune response of said cells to therapy.

### CROSS-REFERENCE TO RELATED APPLICATION

This application is a continuation-in-part of U.S. patent application Ser. No. 15/534037 filed Jun. 8, 2017, the contents of which is incorporated herein by reference.

### FIELD

This specification relates generally to magnetic resonance imaging, and specifically to a system and method for producing image contrasts in magnetic resonance imaging.

### BACKGROUND

In the field of medicine, imaging and image guidance are a significant component of clinical care. From diagnosis and monitoring of disease, to planning of the surgical approach, to guidance during procedures and follow-up after the procedure is complete, imaging and image guidance provides effective and multifaceted treatment approaches, for a variety of procedures, including surgery and radiation therapy. Targeted stem cell delivery, adaptive chemotherapy regimes, and radiation therapy are only a few examples of procedures utilizing imaging guidance in the medical field.

Advanced imaging modalities such as Nuclear Magnetic Resonance (NMR) imaging or Magnetic Resonance Imaging (MRI) as it is commonly known have led to improved rates and accuracy of detection, diagnosis and staging in several fields of medicine including neurology, where imaging of diseases such as brain cancer, stroke, Intra-Cerebral Hemorrhage (ICH), and neurodegenerative diseases, such as Parkinson's and Alzheimer's, are performed. As an imaging modality, MRI enables three-dimensional visualization of tissue with high contrast in soft tissue without the use of ionizing radiation. This modality is often used in conjunction with other modalities such as Ultrasound (US), Positron Emission Tomography (PET) and Computed X-ray Tomography (CT), by examining the same tissue using the different physical principles available with each modality. CT is often used to visualize bony structures, and blood vessels when used in conjunction with an intravenous agent such as an iodinated contrast agent. MRI may also be performed using a similar contrast agent, such as an intravenous gadolinium-based contrast agent which has pharmacokinetic properties that enable visualization of tumors, and breakdown of the blood brain barrier. These multi-modality solutions can provide varying degrees of contrast between different tissue types, tissue function, and disease states. Imaging modalities can be used in isolation, or in combination to better differentiate and diagnose disease.

In neurosurgery, for example, brain tumors are typically excised through an open craniotomy approach guided by imaging. The data collected in these solutions typically consists of CT scans with an associated contrast agent, such as iodinated contrast agent, as well as MRI scans with an associated contrast agent, such as a gadolinium contrast agent. Also, optical imaging is often used in the form of a microscope to differentiate the boundaries of the tumor from healthy tissue, known as the peripheral zone. Tracking of instruments relative to the patient and the associated imaging data is also often achieved by way of external hardware systems such as mechanical arms, or radiofrequency or optical tracking devices. As a set, these devices are commonly referred to as surgical navigation systems.

The link between immunological response imaging and therapy is critical to managing treatment in a number of areas, such as oncology, multiple sclerosis (MS) lesions, stroke penumbra, traumatic brain injury, etc. It is therefore desirable to observe the natural immune response to a tumor or trauma, as well as the immune response being mediated by therapy, for example increased or decreased immune response as a result of tumor or brain injury therapy. Macrophages play a key role in the immunological response; therefore, the ability to image and track macrophage activity in vivo would provide great insight into the immunological response of the body.

MRI is a non-invasive imaging modality that can produce high resolution, high contrast images of the interior of a subject. MRI involves the interrogation of the nuclear magnetic moments of a sample placed in a strong magnetic field with radio frequency (RF) magnetic fields. During MRI the subject, typically a human patient, is placed into the bore of an MRI machine and is subjected to a uniform static polarizing magnetic field B0 produced by a polarizing magnet housed within the MRI machine. Radio frequency (RF) pulses, generated by RF coils housed within the MRI machine in accordance with a particular localization method, are typically used to scan target tissue of the patient. MRI signals are radiated by excited nuclei in the target tissue in the intervals between consecutive RF pulses and are sensed by the RF coils. During MRI signal sensing, gradient magnetic fields are switched rapidly to alter the uniform magnetic field at localized areas thereby allowing spatial localization of MRI signals radiated by selected slices of the target tissue. The sensed MRI signals are in turn digitized and processed to reconstruct images of the target tissue slices using one of many known techniques.

When a substance, such as human tissue is subjected to the static polarizing magnetic field B0, the individual magnetic moments of the spins in the tissue attempt to align with the static polarizing magnetic field B0, but precess about the static polarizing magnetic field B0 in random order at their characteristic Larmor frequency. The net magnetization vector lies along the direction of the static polarizing magnetic field B0 and is referred to as the equilibrium magnetization M0. In this configuration, the Z component of the magnetization or longitudinal magnetization MZ is equal to the equilibrium magnetization M0. If the target tissue is subjected to an excitation magnetic field B1, which is in the x-y plane and which is near the Larmor frequency, the longitudinal magnetization MZ may be rotated, or “tipped” into the x-y plane to produce a net transverse magnetic moment MXY. When the excitation magnetic field B1 is terminated, relaxation of the excited spins occurs, with a signal being emitted that affects the magnitude of radiated MRI signals. The emitted signal is received and processed to form an image.

In particular, when the excitation magnetic field B1 is terminated, the longitudinal magnetization MZ relaxes back to its equilibrium. The spin lattice relaxation time, T1 characterizes the exponentially asymptotic regrowth of the longitudinal magnetization MZ to its equilibrium M0.

The net transverse magnetization MXY decays in exponential fashion as the individual spins begin to de-phase from each other after excitation by the B1 field. The exponential time constant that governs how quickly the transverse magnetization MXY decays is commonly referred to as the transverse relaxation time or the spin-spin relaxation time T2. The transverse relaxation time T2 characterizes how quickly the transverse magnetic moment MXY decays to zero. Both the spin lattice relaxation time, T1 and the transverse relaxation time, T2 are tissue specific, vary with concentration of different chemical substances in the tissue as well as with different microstructural features of the tissue, depend on temperature and the strength of the externally applied magnetic field, B0. Disease or injuries are often conspicuous on MRI due to their effects on tissues, which are observed as differences in image contrast due to changes in the spin lattice relaxation time T1 and/or the transverse relaxation time T2 compared to nearby unaffected tissue.

Like many diagnostic imaging modalities, MRI can be used to differentiate tissue types, e.g. muscles from tendons, white matter from gray matter, and healthy tissue from pathologic tissue. There exist many different MRI techniques, the utility of each depending on the particular tissue under examination. Some techniques examine the rate of tissue magnetization (governed by T1), while other techniques measure the amount of bound water (diffusion imaging) or the velocity of blood flow. Often, several MRI techniques are used together to improve tissue identification. In general, the greater the number of tests available the better chance of producing an accurate diagnosis.

In some instances, exogenous contrast agents (substances which are injected into a person) may be used to emphasize certain anatomical regions. For example, a gadolinium chelate injected into a blood vessel will produce enhancement of the vascular system, or the presence and distribution of leaky blood vessels by its influence on the spin-lattice relaxation of its environment. Iron-loaded stem cells injected into the body and detected by MRI, will allow stem cell migration and implantation in vivo to be tracked, For a contrast agent to be effective, the contrast agent must preferentially highlight one tissue type (diseased vs. normal) or an organ over another. Furthermore, the preferential augmentation of signal (known as image contrast) must be specific to the particular tissue type or cell of interest.

All contrast agents will shorten the T1 and T2 relaxation times of nearby tissue; however, it is useful to subdivide them into two main groups. Group 1: T1 contrast agents, or “positive” agents, decrease T1 relaxation time approximately the same amount as T2 relaxation time, these agents typically produce increased signal intensity (known as positive contrast) within their vicinity in images. Examples of T1 agents are paramagnetic gadolinium- and manganese-based agents. Group 2: These can be classified as T2 contrast agents, or “negative” agents, these agents decrease T2 relaxation time much more so than T1 relaxation time and hence typically result in a reduction of signal intensity in images (negative contrast). Examples of T2 contrast agents are ferromagnetic and superparamagnetic iron oxide based particles, commonly referred to as superparamagnetic iron oxide (SPIO) and ultra-small superparamagnetic iron oxide (USPIO) particles.

The innate ability of an agent to cause T1 relaxation is known as spin-lattice relaxivity, r1 and is a property of the contrast agent itself. The relaxivity of an agent has typical units of mM⁻¹s⁻¹. The rate of relaxation, R1 is defined as R1=1/T1. The relaxation rate of tissue in the vicinity of a contrast agent with relaxivity r1, is given by: R1=R10+r1*[CA], where R10 is the relaxation rate of the tissue in the absence of contrast agent (CA) and [CA] is the local concentration of the contrast agent, usually in mmol/L.

Contrast agents can further be classified as targeted or non-targeted. A targeted contrast agent has the ability to bind to specific molecules of interest. In some cases, the ability of an agent to affect spin-lattice relaxation (i.e. the spin-lattice relaxivity, r1) is greatly enhanced upon binding. For example,_Gadofosveset is a contrast agent that binds to serum albumin in the blood. For many agents (including Gadofosveset), the spin-lattice relaxivity, r1 of the agent in the bound state is also a strong function of the magnetic field strength over certain ranges of strength. When this is the case the contrast agent is said to have T1 (or R1) dispersion.

Delta relaxation enhanced magnetic resonance (DREMR), generally referred to as field-cycled relaxometry or field-cycled imaging is an MRI technique that relies on using underlying tissue contrast mechanisms that vary with the strength of the applied magnetic field in order to generate novel image contrasts. To achieve DREMR contrast, the main magnetic field is varied as a function of time during specific portions of an MRI pulse sequence. A field-shifting electromagnet coil is used to perform the field variation. The DREMR method exploits the difference in the R1 dispersion properties (variation of r1 with field strength) of targeted spin-lattice contrast agents in the bound and unbound states in order to obtain an image that contains signal only from tissues enhanced by contrast agent that is in the bound state, while suppressing signal from tissues in the vicinity of unbound contrast agent or tissues, which do not contain the agent at all.

Relatively recently, iron oxide nanoparticles have become the preferred approach to track macrophage activity within the body. This is achievable because macrophages have naturally high endocytosis activity and hence will internalize the contrast agent after it has been injected into the subject. These cells also hone in on areas of inflammation caused by diseases or injury as part of the innate immune response mechanism in humans and animals. Once a substantial amount of contrast agent has accumulated in the macrophage and/or a substantial amount of macrophages containing lesser amounts of contrast agent have accumulated, the MRI signal will be decreased in the tissues of the immediate area due to the shortening of T2 relaxation time caused by the presence of the contrast agent. This change in signal can be detected by use of subtraction between pre- and post-injection MRI images. Those areas with significant changes in signal after subtraction are indicative of the presence of macrophages containing iron oxide nanoparticles and hence regions of inflammation.

There are a few problems with the above approach. For example, the approach depends on a subtraction between pre- and post-injection images. These images must be taken at different times often on different days and tissue may move between scans causing subtraction artifacts. Attempts to avoid this dependence on a pre-injection scan may seek to simply monitor locations where there is signal dropout. However, signal dropout can be caused by other, non-contrast related, phenomena; for example, susceptibility differences between tissues or air moving through the digestive system. If there is already signal dropout present due to other phenomena, additional signal dropout cannot be detected. This, in turn, points to a further problem with the aforementioned technique to monitor macrophage activity: once enough contrast agent has accumulated to produce adequate signal dropout, additional accumulation cannot be detected. This leads to a maximum concentration of contrast agent that can be detected within a certain region, thereby making the above-mentioned method to track macrophage activity non-quantifiable.

### SUMMARY

The present inventor has found that contrast agents selected from the group consisting of superparamagnetic iron oxide (SPIO) and ultra-small superparamagnetic iron oxide (USPIO) particles possess a strong relaxivity, r1, dependence on the strength of the magnetic field. Therefore, the DREMR method can be used to obtain positive contrast images that contain signal specifically where these iron-oxide-based contrast agents have accumulated.

An aspect of the specification provides a method of imaging soft tissue comprising: administering a contrast agent comprising superparamagnetic iron oxide (SPIO) nanoparticles to soft tissue; and imaging a region of interest associated with the soft tissue using DREMR imaging to obtain positive contrast images due to the presence of SRIO nanoparticles, possessing T1 dispersion.

Another aspect of the specification provides A contrast agent selected from the group consisting of ferumoxytol, ferucarbotran, ferumoxide, FeRex, and Ferumoxtran-10 for use in generating positive contrast images using DREMR imaging due to T1 dispersion of the contrast agent.

The present invention can be used in a number of applications, including locating reactive brain cells (e.g. astrocytes and macrophages) in or at the margins of brain tumors; intra-operative surgical resection assessment; and screening for tumor metastases.

### BRIEF DESCRIPTIONS OF THE DRAWINGS

Embodiments are described with reference to the following figures, in which:

FIG. 1 shows a block diagram of functional subsystems of a delta relaxation enhanced magnetic resonance (DREMR) imaging system in accordance with an implementation.

FIGS. 2A and 2B show successive single field shift DREMR sequences.

FIG. 3 shows an example “positive” field-shift image, “negative” field-shift image, subsequent subtracted image (positive field-shift image minus negative field-shift image), intensity correction image, and the final normalized subtracted image.

FIG. 4 is a flowchart showing steps for using the DREMR imaging method of

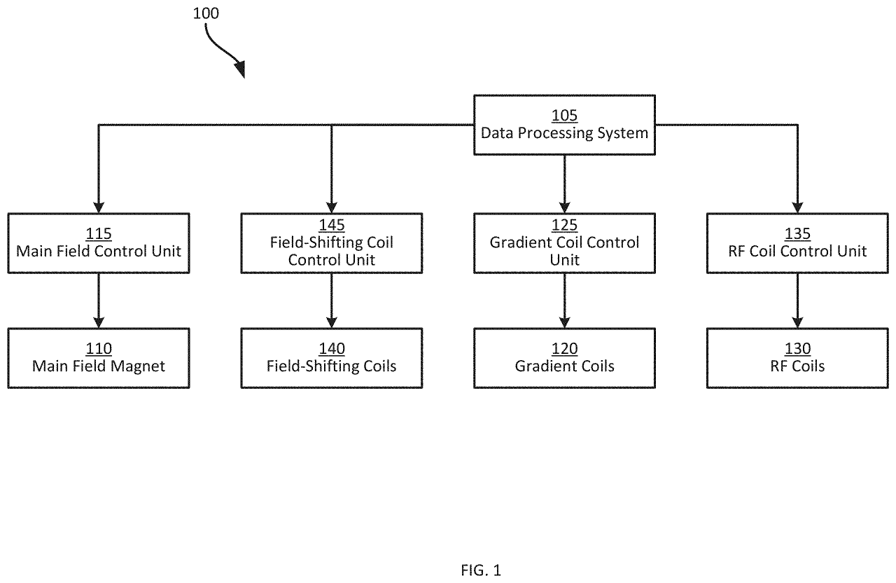

FIGS. 1-3 to visualize macrophage activity and response to therapy after administration of iron oxide based contrast agents.

FIG. 5 is a graph comparing the relaxivity data for Feraheme (and iron oxide based contrast agent) and Dotarem (Gadoterate Meglumine; a clinical paramagnetic contrast agent).

FIG. 6 shows T1-weighted and DREMR images of in-vivo mouse tissue with a xeno-grafted human breast cancer tumour.

FIG. 7 shows a double inversion dreMR sequence.

FIG. 8 shows nuclear magnetic relaxation dispersion (NMRD) data for ferucarbotran.

### DETAILED DESCRIPTION

Various embodiments and aspects of the disclosure will be described with reference to details discussed below. The following description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms “comprises” and “comprising” are to be construed as being inclusive and open ended, and not exclusive. Specifically, when used in the specification and claims, the terms “comprises” and “comprising” and variations thereof mean the specified features, steps or components are included. These terms are not to be interpreted to exclude the presence of other features, steps or components. The terms “consist of” and “consisting of” are to be construed as to be exhaustive, meaning, the specified features, steps or components are included and other features, steps, or components are excluded, except for those features, steps or components that are or may be inherently present in the recited elements. The terms “consists essentially of” or “consisting essentially of” shall be construed to mean “consists of” or “consists essentially of” the recited elements and additional elements (e.g. features, steps, or components) that would or do not materially affect the basic and novel properties of the invention, in accordance with the interpretation applied under U.S. patent law. By “basic and novel properties” is meant the ability to obtain positive contrast images using the DREMR method herein described.

As used herein, the term “exemplary” means “serving as an example, instance, or illustration,” and should not be construed as preferred or advantageous over other configurations disclosed herein.

Referring to FIG. 1, a block diagram of a delta relaxation magnetic resonance imaging (DREMR) system, in accordance with an example implementation, is shown at 100. The example implementation of the DREMR system indicated at 100 is for illustrative purposes only, and variations including additional, fewer and/or varied components are possible. Traditional magnetic resonance imaging (MRI) systems represent an imaging modality which is primarily used to construct pictures of nuclear magnetic resonance (MR) signals from protons such as hydrogen atoms in an object. In medical MRI, typical signals of interest are MR signals from water and fat, the major hydrogen containing components of tissues. DREMR systems use field-shifting magnetic resonance methods in conjunction with traditional MRI techniques to obtain images with different contrast than is possible with traditional MRI, including molecularly-specific contrast.

As shown in FIG. 1, the illustrative DREMR system 100 comprises a data processing system 105. The data processing system 105 can generally include one or more output devices such as a display, one or more input devices such as a keyboard and a mouse as well as one or more processors connected to a memory having volatile and persistent components. The data processing system 105 can further comprise one or more interfaces adapted for communication and data exchange with the hardware components of MRI system 100 used for performing a scan.

Continuing with FIG. 1, the exemplary DREMR system 100 can also include a main field magnet 110. The main field magnet 110 can be implemented as a permanent, superconducting or a resistive magnet, for example. Other magnet types, including hybrid magnets suitable for use in the DREMR system 100 will be known to a person of skill in the art and are contemplated. The main field magnet 110 is operable to produce a substantially uniform main magnetic field having strength B0 and a direction along an axis. The main magnetic field is used to create an imaging volume within which desired atomic nuclei of an object, such as the protons in hydrogen within water and fat, are magnetically aligned in preparation for a scan. In some implementations, as in this example implementation, a main field control unit 115 can communicate with data processing system 105 for controlling operation of the main field magnet 110.

The DREMR system 100 can further include gradient magnets, for example gradient coils 120 used to produce deliberate variations in the main magnetic field (B0) along, for example, three perpendicular gradient axes. The size and configuration of the gradient coils 120 can be such that they produce a controlled and uniform linear gradient. For example, three paired orthogonal current-carrying coils located within the main field magnet 110 can be designed to produce desired linear-gradient magnetic fields. The variation in the magnetic field permits localization of image slices as well as phase encoding and frequency encoding spatial information.

The magnetic fields produced by the gradient coils 120, in combination and/or sequentially, can be superimposed on the main magnetic field such that selective spatial excitation of objects within the imaging volume can occur. In addition to allowing spatial excitation, the gradient coils 120 can attach spatially specific frequency and phase information to the atomic nuclei placed within the imaging volume, allowing the resultant MR signal to be reconstructed into a useful image. A gradient coil control unit 125 in communication with the data processing system 105 can be used to control the operation of the gradient coils 120.

The DREMR system 100 can further comprise radio frequency (RF) coils 130. The RF coils 130 are used to establish an RF magnetic field with strength B1 to excite the atomic nuclei or “spins” within an object being imaged. The RF coils 130 can also detect signals emitted from the “relaxing” spins within the object. Accordingly, the RF coils 130 can be in the form of separate transmit and receive coils or a combined transmit and receive coil with a switching mechanism for switching between transmit and receive modes.

The RF coils 130 can be implemented as surface coils, which are typically receive-only coils and/or volume coils which can be receive-and-transmit coils. The RF coils 130 can be integrated in the main field magnet 110 bore. Alternatively, the RF coils 130 can be implemented in closer proximity to the object being imaged, such as a head, and can take a shape that approximates the shape of the object, such as a close-fitting helmet. An RF coil control unit 135 can be used to communicate with the data processing system 100 to control the operation of the RF coils 130.

In order to create a contrast image in accordance with field-shifting techniques, DREMR system 100 can use field-shifting electromagnets 140 while generating and obtaining MR signals. The field-shifting electromagnets 140 can modulate the strength of the main magnetic field. Accordingly, the field-shifting electromagnets 140 can act as auxiliary to the main field magnet 110 by producing a field-shifting magnetic field that augments or perturbs the main magnetic field. A field-shifting electromagnet control unit 145 in communication with the data processing system 100 can be used to control the operation of the field-shifting electromagnets 140.

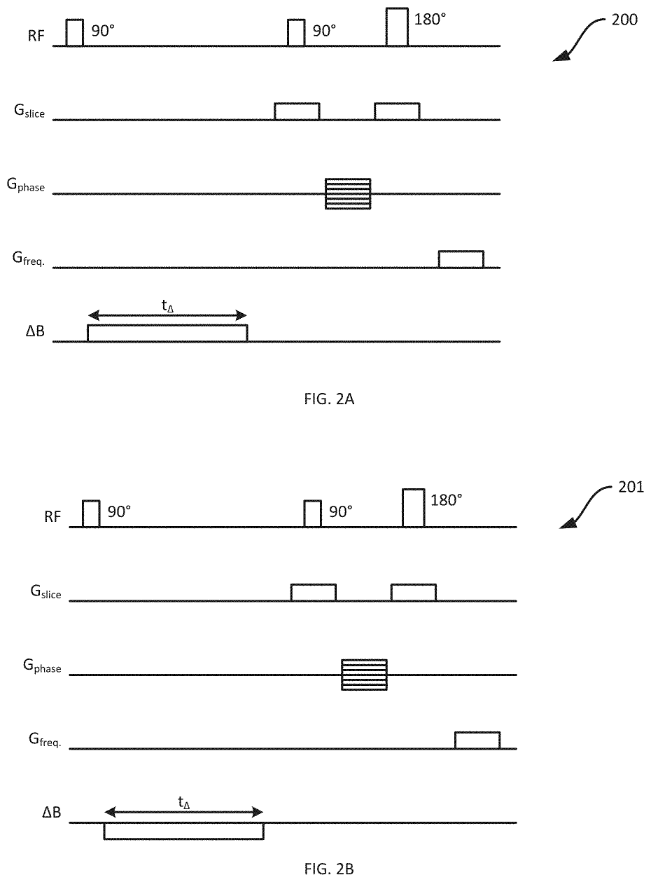

There are many techniques for obtaining images that will produce contrast related to the T1 dispersion of tissue using the DREMR system 100. To provide an illustration of this, simplified operations for obtaining an image with contrast specific to the change in relaxation rate (1/T1) between two distinct polarizing magnetic field strengths will be described as a non-limiting example. Referring now to FIG. 2A and FIG. 2B, illustrative DREMR pulse sequences are shown. Specifically, timing diagrams for the example pulse sequences are indicated. The timing diagrams show pulse or signal magnitudes, as a function of time, for transmitted (RF) signal, magnetic field gradients (Gslice, Gphase, and Gfreq), and field-shifting signal (ΔB). The RF pulses can be generated by the transmit aspect of the RF coils 130. The waveforms for the three gradients can be generated by the gradient coil control unit 125 and the gradient coils 120 produce the corresponding magnetic fields. The waveform for the field-shifting signal can be generated by the field-shifting coil control unit 145 and the field-shifting electromagnet 140 produces the corresponding magnetic field. The precise timing, amplitude, shape, and duration of the pulses or signals may vary for different imaging techniques. For example, the field-shifting signal may be applied for a shorter or longer duration or at a larger or smaller amplitude such that the image contrast due to T1 dispersion is optimized.

Referring now to FIG. 2A, the first event to occur in pulse sequence 200 can be to apply an RF pulse such that it produces a 90 degree rotation of the magnetization from the Z-axis (the direction of the main magnetic field) into the XY-plane (the plane of detection of the receiver coils). This has the effect of making the magnetization along the Z-axis, denoted MZ, zero. Once the first 90 degree RF pulse has finished, the field-shifting electromagnet 140 can be turned on for a time period of t_{Δ}. In this first sequence the field-shifting electromagnet 140 is turned on such that the field that is produced is additive to (i.e. increases) the main magnetic field B0. Once the field-shifting electromagnet 140 is turned off the pulse sequence can continue with a particular imaging sequence. In this example implementation, the imaging sequence that is used is a spin-echo sequence; however, other pulse sequence strategies for image acquisition can be used. For an example SPIO contrast agent such as ferumoxytol, the spin-echo sequence may not be desirable due to the very short spin-spin tissue relaxation caused by the large r2 relaxivity of SPIO particles. For SPIO particle imaging, ultrashort echo time (UTE) sequences may be preferable, to preserve signal (for example, a fast, spoiled gradient-recalled echo sequence, optimized for very short Time-to-Echo (TE) values of less than 2 ms). This is the case for imaging with either field shift (i.e. for either FIG. 2A or 2B).

Referring now to FIG. 2B, once again the first event to occur in pulse sequence 201 can be to apply an RF pulse such that it produces a 90 degree rotation of the magnetization from the Z-axis (the direction of the main magnetic field) into the XY-plane (the plane of detection of the receiver coils). This has the effect of making the magnetization along the Z-axis, denoted MZ, zero. Once this first 90 degree RF pulse has finished, the field-shifting electromagnet 140 can be turned on for a time period of t_{Δ}, in this second sequence the field-shifting electromagnet is turned on such that the field that is produced is subtracted from (i.e. decreases) the main magnetic field B0. Once the field-shifting electromagnet 140 is turned off the pulse sequence can continue with a particular imaging sequence. In this example implementation, the imaging sequence that is used is a spin-echo sequence.

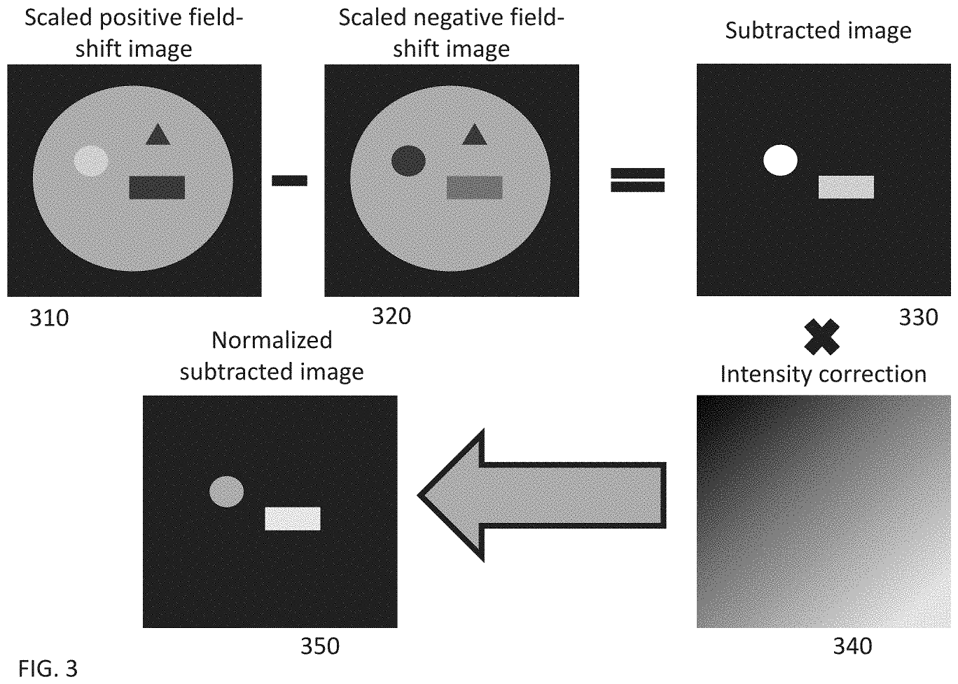

Referring now to FIG. 3, there is an image corresponding to the positive field-shift sequence from FIG. 2A denoted “scaled positive field-shift image” at 310, the word “scaled” has been added to the description of this image to indicate the multiplication by a scalar factor needed prior to subtraction. Similarly, there is an image corresponding to the negative field-shift sequence from FIG. 2B denoted “scaled negative field-shift image” at 320, once again the word “scaled” has been added to the description to indicate the multiplication by a scalar factor that is needed prior to subtraction. These two images can be subtracted from each other to produce a “subtracted image” as indicated at 330. Due to inhomogeneities in the polarizing field that is produced by the field-shifting electromagnet 140 (i.e. the field-shift in one region of space may be slightly larger than the field-shift in another region of space), the subtracted image must be multiplied by an intensity correction image (340) on a pixel-by-pixel basis. The value assigned to each pixel of the intensity correction image 340 can be calculated, for example, based on the difference between (i) the field-shift caused by the field-shifting coils 140 at the relevant pixel location and (ii) the field-shift at iso-center (the center of the imaging region). After multiplying the subtracted image 330 by the intensity correction image 340 the result is the “Normalized subtracted image at 350. It is important to note that the field-shift images 310 and 320 do not necessarily need to be “positive” (i.e. adding to the main field) and “negative” (i.e. subtracting from the main field). The field-shifting images 310 and 320 must only be captured at two distinct polarizing fields.

According to the present invention, MRI contrast agents, such as SPIOs and USPIOs are injected into tissue. The contrast agent is subsequently engulfed by inflammatory cells (macrophages), with the result that MRI signal due to T1 dispersion (i.e. signal produced using the DREMR methodology described above) correlates with macrophage density.

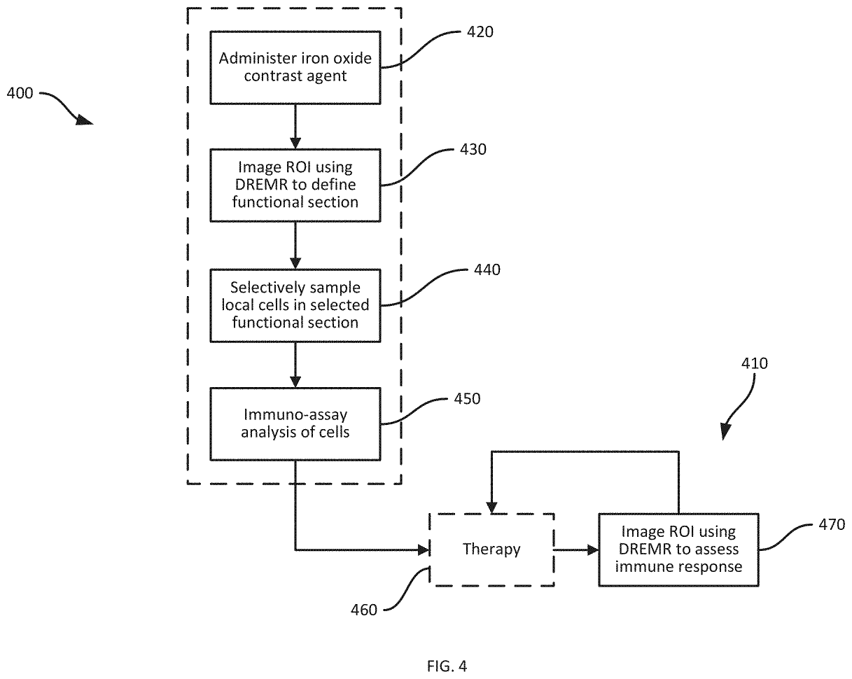

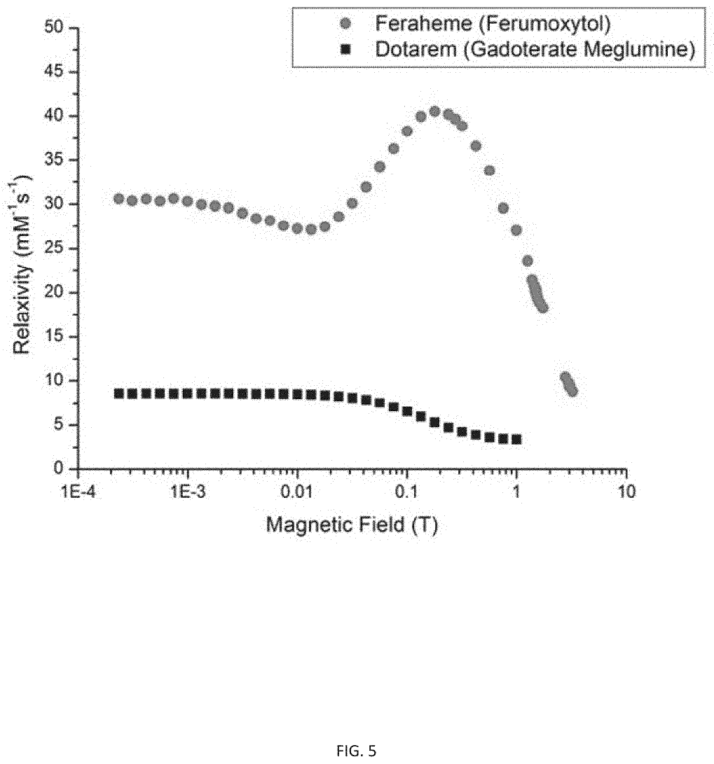

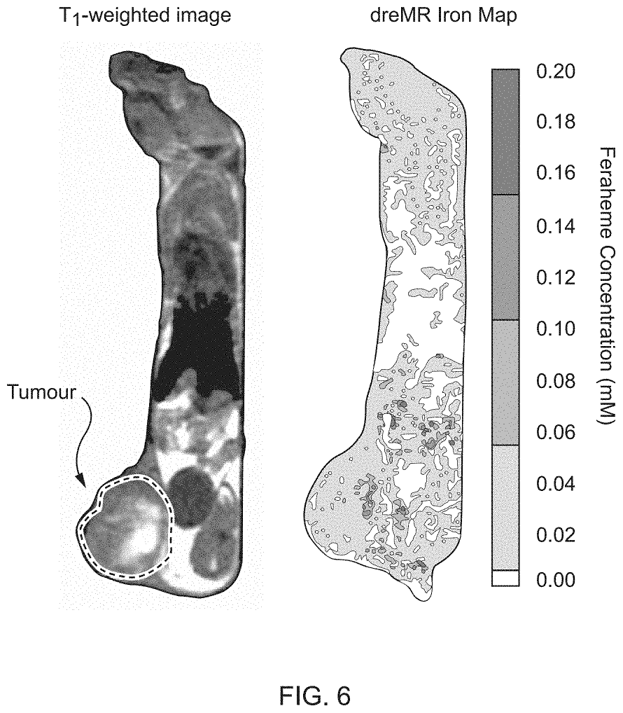

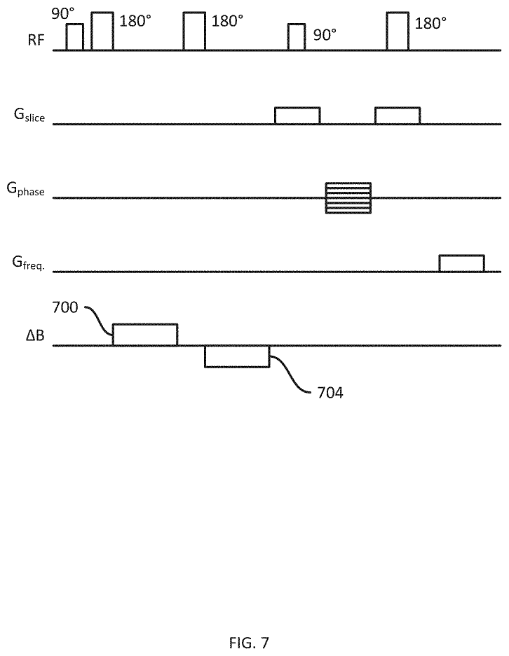

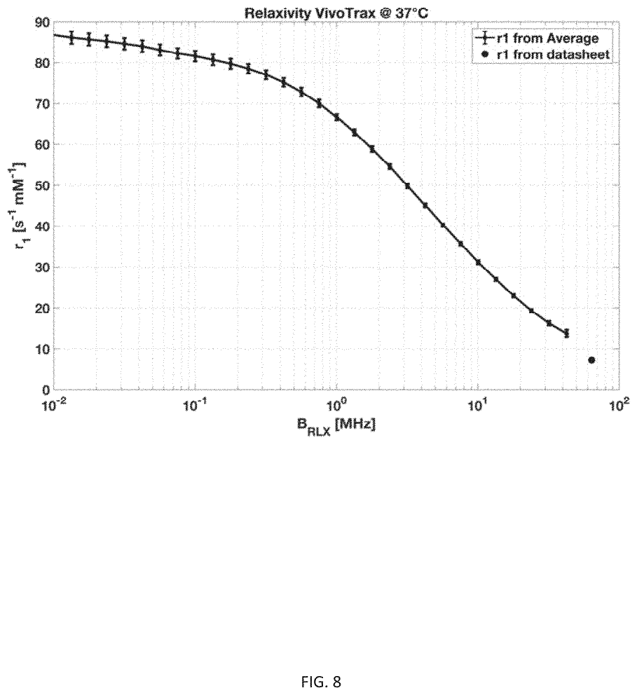

According to one aspect of the present invention, the DREMR imaging system of FIGS. 1-3 may be used to visualize immune response by administering SPIO or USPIO contrast agents, according to the steps set forth in FIG. 4, wherein part 400 shows steps for visualizing the natural immune response of tissue in a region of interest (ROI), and part 410 shows steps of visualizing the immune response being mediated by therapy (e.g. increased immune response resulting from immunologically responsive tumor therapy, or decreased immune response due to brain (or other) injury therapy. The system discussed herein may also be used to visualize the spatial distribution of iron-labeled cells injected for cell therapies, e.g. to track the travel of such cells after injection and determine whether the cells are present at a desired target site or the like.

At 420, a contrast agent selected from the group consisting of superparamagnetic iron oxide (SPIO) and ultra-small superparamagnetic iron oxide (USPIO) is administered (e.g. via injection). The ROI is imaged, using DREMR imaging, to determine the concentration of the contrast agent. A functional section is then identified where the concentration of contrast agent is above a predetermined threshold, as the contrast agent (carried by macrophages) will accumulate in areas of inflammation indicative of a tumor or area of trauma. In this example implementation, the term “functional section” is defined as an area within a region of interest where the signal produced by the DREMR methodology is larger than a pre-defined threshold. However, it is important to note that the criteria for a functional section may change for other implementations, such as being located in the immediate vicinity of a known region of trauma.

Referring back to this example implementation, to quantify the concentration of contrast agent in the ROI, external reference standards of the contrast agent in known concentrations can be used. These reference standards have imaging properties which mimic the tissue in the ROI. That is, the reference standards contain materials whose spin-lattice and spin-relaxation properties are similar to those of the tissue in the ROI. In this example, the reference standard can be made using a gel, such as agar, whose concentration is adjusted to obtain these properties. A series of vials of these gels containing different known concentrations of contrast agent (i.e. the above-mentioned SPIO or USPIO) is placed near the anatomical region of interest and included within the imaging field-of-view. In other embodiments, the reference standards can be made of other gels, e.g. agarose or the like.

A selective analysis is then performed on the functional section, at steps 440 and 450. In one embodiment, at 440, local cells within the functional section are selectively sampled (e.g. via biopsy) and then, at 450, immuno-assay analysis is conducted on the sampled cells in the selected area (e.g. to identify the natural targets of the tumor). In alternative embodiments, a selective analysis is performed which compares cells within the functional section with cells of known types stored within a database or informatics system.

Then, at 460, appropriate therapy is performed based on the diagnostic process of part 400. At 470, the ROI is again imaged using DREMR imaging in the same manner as described above with reference to numeral 430, to assess immune response and adjust therapy 460 for enhancing the immuno-response to these cells. Note that the actual therapy 460 does not form part of the diagnostic method of the present invention.

According to further aspects of the invention, several applications of the system and method set forth above are contemplated.

In one application, DREMR imaging is performed at 430 to locate reactive brain cells (e.g. astrocytes and macrophages) in or at the margins of brain tumors and in locations not otherwise identified by MR imaging methods. Using the location of reactive brain cells identified in this manner, therapy 460 may be specifically targeted (e.g. to guide margins of tumor resection, guide injection of immuno-response specific therapeutic agents, guide tissue biopsy, etc.).

In a surgical application, since SPIOs have been demonstrated to accumulate in areas of active macrophages over the course of many hours and remain detectable for 2-5 days post injection, DREMR imaging may be performed intra-operatively at 470 to assess the extent of surgical resection. Other intra-operative MR imaging methods which rely on tissue contrast mechanisms may become intra-operatively compromised (e.g. T2-mediated contrast that can be confounded by bleeding or fluid accumulation in the resection cavity; gadolinium contrast-enhanced imaging which can be confounded by gadolinium leaking into the resection cavity; and other acute vascular permeability changes due to the surgical process, not related to tumor vascularity). According to an aspect of the invention, intra-operative DREMR imaging at 470 may be used to detect SPIOs that have been administered pre-operatively at 420, to visualize residual reactive tissue targets for further resection.

In another diagnostic application, DREMR imaging in accordance with 400 and 410 may be used to screen for tumor metastases (e.g. by locating SPIOs that have accumulated in areas of active tumors).

One example of an iron-oxide-based contrast agent useful in the context of the present invention is ferumoxytol (sold as Feraheme™ by AMAG Pharmaceuticals). Ferumoxytol is a clinically approved treatment for chronic kidney disease (CKD), and is comprised of an emulsion of iron oxide nanoparticles. Published manufacturer's data shows that ferumoxytol has a mean hydrodynamic diameter of 30 nm, an r1 relaxivity of 38 s mM⁻¹s⁻¹, and an r2 relaxivity of 83 mM⁻¹s⁻¹ at 0.94 T and at 37 C.

The present inventor has found that ferumoxytol has the relaxivity data shown in FIG. 5. FIG. 5 shows the relaxivity of ferumoxytol compared with that of Dotarem (gadoterate meglumine), a clinical paramagnetic contrast agent. The T1 dispersion of ferumoxytol is much greater than that of gadoterate meglumine at magnetic field strengths greater than 0.01 T. The amount of T1 dispersion and its dependency on magnetic field strength depends on the particular SPIO particle, particularly, its size.

FIG. 6 is a cropped image of an in-vivo image of a mouse with xeno-grafted human breast cancer tumour. Prior to taking this image, Feraheme was injected into the mouse under anesthesia at a dose of 0.5 mmol [Fe]/kg. The Feraheme was injected through the tail vein and the mouse was returned to a cage 24 hours prior to imaging. The mouse was then anesthetized using 2% Isoflurane after induction at 4%, and then placed on a heated pad in MRI RF coil. The Feraheme concentration was quantified in mmol/L using the dreMR method and calibration vials described above. The calibration vials are not shown in the cropped image but exist outside the boundaries.

In this example involving ferumoxytol, the dreMR pulse sequence included UTE image acquisition, i.e. fast spoiled-gradient recalled-echo or radial/spiral imaging. A suitable field strength, B0, can be selected based on the SPIO or targeted contrast agent used. The relaxivity data for ferumoxytol shows that an appropriate field strength would be 0.01 T to 0.3 T or B0>1.0 T.

A particular advantage to using Feraheme in the context of the present invention is that Feraheme is approved for human use, e.g. for the treatment of chronic kidney disease (CKD) in some jurisdictions. Other SPIO or USPIO particles that are suitable for use in the above systems and methods may not have such regulatory approval, which may present an obstacle to their use for injection as discussed above.

In order for ferumoxytol to be used successfully in the above method, the cells must successfully internalize the SPIO without causing cell death. The primary field strength must be chosen where the r1 dispersion curve has a large slope: (for Feraheme 0.01 T to 0.3 T and >1.0 T). Also, as previously mentioned, quantitative imaging is possible only with special calibration or reference standards that must be included within the imaging the region of interest. Imaging of the SPIO particles must also include short time-to-echo imaging, that is ultra-short echo imaging acquisition. This is because SPIO particles are very efficient R2 agents. As a result, the spin-spin relaxation of neighbouring tissues is very fast requiring short echo-time imaging (TE <2 ms etc.) Spin-echo image acquisition or other imaging with longer TE values result in signals with very poor contrast due to the resulting loss of signal.

Other pulse sequences may be employed, in addition to those discussed above in connection with FIGS. 2A and 2B. FIGS. 2A and 2B depict successive single field shift DREMR sequences. That is, FIGS. 2A and 2B illustrate a sequence that is repeated with both positive (FIG. 2A) and negative (FIG. 2B) field shifts (ΔB) and then the images can be subtracted after normalization for magnetization differences (for example, see: Magn Reson Med 61:796-802 (2009)). FIG. 7 illustrates a double inversion recovery (DIR) DREMR sequence, in which two distinct field shifts 700 and 704 are employed. This sequence has specially chosen values for the durations of the field shifts which are specific to the relaxivities of the tissues which are being suppressed and the relaxation of the tissues or proteins which are being enhanced by the SPIO particles, Relaxation data for murine tissues can be found in the publication: NMR Biomed 30:e3789 (2017). A system of equations known as the Bloch Equations are numerically solved to determine the individual durations of the field shifts 700 and 704 given the nominal imaging field strength, tissue relaxation and SPIO relaxivity data. DREMR imaging data can be quantified by including solutions of known contrast agent concentration and target protein (if relaxivity of agent changes upon binding).

At present the imaging portion of these sequences (that portion of the sequence beginning with the final 90° pulse) is a fast spin-echo sequence, However, a fast gradient recalled echo sequence may be more appropriate for iron imaging. For SPIO particle imaging, short TEs (<2 ms) are required due to the short T2*. In either case, the imaging parameters, TE, TR, flip angle etc. are chosen for T1 weighting of the image (TE<2 ms and shortest TR compatible with other imaging parameters of the sequence).

Although the applications set forth in detail above are directed at managing immune response in neurological treatment such as treating brain tumors and injuries, the DREMR imaging with SPIO contrast enhancement as set forth herein may be used in the identification and/or treatment of any disease, disorder or condition involving inflammation, injury, and/or passive accumulation of macrophages, e.g. MS lesions, stroke, other conditions involving vascular damage, etc.

The SPIO or USPIO can also be used to track labelled stem or other therapeutic cells for cell therapies, e.g. by co-incubating stem cells or other therapeutic cells with the SPIO or USPIO in vitro and then injecting the labelled cells into a patient. The labelled cells can then be tracked in vivo with DREMR. In one embodiment of this method, after cell culture and expansion, Feraheme (50 μg Fe/mL) can be added to the cell culture medium and allowed to co-incubate with the cells overnight. Then, the cells can be washed with a phosphate-buffered saline and harvest by trypsin-EDTA digestion. The labelled cells can then be injected into an animal. (e.g. foot pad injection, cardiac injection or intravenous injection depending on experiment) and then tracked in vivo using the present dreMR method and system.

In an alternative embodiment, Feraheme concentration can be determined in vivo by relaxometry mapping R1 at two or more field strengths using fast field-cycling imaging. The use of external reference vials can be avoided in this manner, at the cost of increased imaging time in comparison to DREMR imaging.

In a further alternative embodiment, Feraheme concentration can be determined using a “subtraction method” discussed above in connection with FIGS. 2A, 2B, and 3. In yet another embodiment, Feraheme concentration can be measured using a “double inversion” method illustrated in FIG. 7. To implement the double-inversion sequence, knowledge of the r1 dispersion of the contrast agent and the R1 dispersion of tissues are required. R1 dispersion data for murine tissues (human tissues have similar values) are available, for example, in: Araya Y T, Martinez-Santiesteban F, Handler W B, Harris C T, Chronik B A, and Scholl T J, Nuclear magnetic relaxation dispersion of murine tissue for development of T1 (R1) dispersion contrast imaging. NMR in Biomedicine, 2017. 30(12): p. e3789-n/a. As noted earlier, the durations of the magnetic field-shifts 700 and 704, as well as their relative amplitudes, are calculated from the Bloch Equations, which govern the evolution of the longitudinal MZ and transverse MXY magnetizations. Once a contrast agent relaxivity is measured, these data can be used for input into the Bloch Equation calculation and field shifts and durations determined for best imaging contrast of the SPIO particle or other DREMR contrast agent.

The double inversion DREMR sequence has specially chosen values for the durations of the field shifts which are specific to the relaxivities of the tissues which are being suppressed and the relaxation of the tissues or proteins which are being enhanced by the SPIO particles. Relaxation data for murine tissues can be found in the publication: NMR Biomed 30:e3789 (2017). It is necessary to numerically solve the above-mentioned Bloch Equations to determine the individual durations of the field shifts given the nominal imaging field strength, tissue relaxation and SPIO relaxivity data. DREMR imaging data can be quantified by including solutions of known contrast agent concentration and target protein (if relaxivity of agent changes upon binding).

At present the imaging portion of these sequences (that portion of the sequence beginning with the final 90° pulse) is a fast spin-echo sequence. However, a fast gradient recalled echo sequence would likely be more appropriate for iron imaging. For SPIO particle imaging, short TEs (<2 ms) are required due to the short T2*. In either case, the imaging parameters, TE, TR, flip angle etc. are chosen for T1 weighting of the image (TE <2 ms and shortest TR compatible with other imaging parameters of the sequence).

Either of the aforementioned subtraction method and the double inversion method could use different image acquisition strategies (i.e. spin-echo, gradient-recalled echo, radial or spiral k-space acquisition, etc.).

Other contrast agents are contemplated as being useful within the context of the present invention. These include: ferucarbotran (sold as VivoTrax™ by Magnetic Insight and sold as Resovist™ by Bayer Schering Pharam). FIG. 8 illustrates nuclear magnetic relaxation dispersion (NMRD) data for ferucarbotran, which the present inventor measured for magnetic fields up to 1 T. The data shows that ferucarbotran is a suitable DREMR contrast agent below field strengths of 1 T, and is particularly suitable at 0.5 T. Manufacturer's data for ferucarbotran indicates an r1 of 7.2±0.1 mM⁻¹s⁻¹ (1.5 T and 37° C.), and an r2 of 82.0±6.2 mM⁻s⁻¹ (1.5 T and 37° C.).

Other examples of SPIOS for use in the above procedures include ferumoxide (sold as Feridex™ by AMAG Pharmaceuticals). The published r1 and r2 relaxivities are 4.7 and 41 mM⁻¹s⁻¹ respectively. Further examples include FeREX™ and FerroTRACK™ (sold by BioPal); Ferumoxtran-10 (AMI-227™; Combidex™ sold by AMAG Pharma; Sinerem™ sold by Guerbet). Manufacturer-published r1 and r2 relaxivities of Combidex/Sinerem are 10 and 60 mM⁻¹sec⁻¹ respectively,

SPIO particles that have been demonstrated to be capable of being taken up by macrophages are disclosed in the articles, Macrophage endocytosis of superparamagnetic iron oxide nanoparticles: mechanisms and comparison of ferumoxides and ferumoxtran-10, Invest Radiol. 2004 January; 39(1):56-63. In this article, the authors conclude, “Competition experiments indicate that the cellular uptake of Ferumoxides involves scavenger receptor SR-A-mediated endocytosis. The comparison between Ferumoxides and Ferumoxtran-10 confirms that macrophage uptake of iron oxide nanoparticles depends mainly on the size of these contrast agents.” See also: Mechanism of Cellular Uptake and Impact of Ferucarbotran on Macrophage Physiology; Yang C Y, Tai M F, Lin C P, Lu C W, Wang J L, et al. (2011) Mechanism of Cellular Uptake and Impact of Ferucarbotran on Macrophage Physiology. PLOS ONE 6(9): e25524. https://doi.org/10. 1371/journal, pone.0025524

SPIO and USPIO particles can be modified by conjugation with peptides, which bind to specific cell membrane proteins or other proteins in the blood. This could enhance the accumulation of SPIO particles in a particular tissue without macrophage intervention. The SPIO particles could also be modified by adding chromophores or fluorescent dyes, which would make them visible by an optical imaging modality. This would produce a dual-modality contrast agent for dreMR/MRI and perhaps fluorescence imaging for in shallow tissue (i.e. superficial tumours or skin cancers, esophageal cancers etc.).

For Magnetic Particle Imaging (MPI), ferucarbotran can be used for therapy. MPI allows for inductive heating of the SPIO. This can be very selective in volume. Something like this might be possible with dreMR for localization of the SPIO particles and an auxiliary magnetic field coil for inductive heating to elicit therapy. MR thermometry could be used to monitor the therapy and predict treatment outcomes.

Certain advantages of the systems and methods discussed above will now be apparent. For example, the use of SPIO or USPIO contrast agents in conjunction with DREMR imaging can obviate the need for pre-injection and post-injection imaging. That is, the presence of the contrast agent does not need to be determined by comparing pre- and post-injection images, but can instead by quantified within a single imaging session (e.g. using the above-mentioned calibration vials).

The scope of the claims should not be limited by the embodiments set forth in the above examples, but should be given the broadest interpretation consistent with the description as a whole.

1. A method of imaging soft tissue comprising:
administering a contrast agent comprising superparamagnetic iron oxide (SPIO) nanoparticles to soft tissue; and
imaging a region of interest associated with the soft tissue using DREMR imaging to obtain positive contrast images due to the presence of SPIO nanoparticles, possessing T1 dispersion.

2. The method of claim 1, wherein the contrast agent is selected from the group consisting of ferumoxytol, ferucarbotran, ferumoxide, FeRex, and Ferumoxtran-10.

3. The method of claim 2, wherein an amount of contrast agent in the region of interest is determined by comparison to a reference standard having imaging properties that mimic the region of interest.

4. The method of claim 3, further comprising:
placing at least one reference standard in an imaging field of view prior to the imaging of the region of interest.

5. The method of claim 2, wherein an amount of contrast agent in the region of interest is determined by using a double inversion recovery (DIR) DREMR imaging method using the R1 relaxivity of the contrast agent and the R1 relaxivity of the tissue in the region of interest or by fast field-cycling relaxometric imaging.

6. The method of claim 1, wherein the contrast agent is ferumoxytol.

7. The method of claim 6, wherein the imaging comprises ultra-short time to echo (UTE) imaging sequences.

8. The method of claim 7, wherein said UTE imaging sequences are selected from the group consisting of fast spoiled-gradient recalled-echo or radial/spiral imaging sequences.

9. The method of claim 7, wherein the main field strength B0 used in said imaging is from 0.01 T to 0.4 T or ≥1.0 T.

10. The method of claim 6 for use in active labelling of cells, wherein the method comprises incubating cells in vitro with ferumoxytol to form incubated cells, injecting the incubated cells into a subject, and tracking the distribution of the incubated cells in vivo using the DREMR imaging.

11. The method of claim 6 for use in passive labelling of cells, wherein the method comprises injecting ferumoxytol into a subject and tracking the distribution of ferumoxytol in vivo using the DREMR imaging.

12. The method of claim 1, wherein the contrast agent is ferucarbotran,

13. The method of claim 12, wherein the main field strength B0 used in said imaging is from 0.01 T to 1.0 T.

14. The method of claim 13, wherein B0 is about 0.5 T.

15. A contrast agent selected from the group consisting of ferumoxytol, ferucarbotran, ferumoxide, FeRex, and Ferumoxtran-10 for use in generating positive contrast images using DREMR imaging due to T1 dispersion of the contrast agent.

16. The contrast agent of claim 15, selected from ferumoxytol and ferucarbotran.

17. The contrast agent of claim 15, wherein the contrast agent is ferumoxytol.
